(19) United States
(12) Patent Application Publication
(10) Pub. No.: US 2024/0130852 A1
(43) Pub. Date: Apr. 25, 2024

(54) HAND OPERATED INTRAOCULAR FLUID DELIVERY DEVICE

(71) Applicant: Nathaniel R. Collins, Monrovia, CA (US)

(72) Inventor: Nathaniel R. Collins, Monrovia, CA (US)

(21) Appl. No.: 18/489,636

(22) Filed: Oct. 17, 2023

Related U.S. Application Data
(60) Provisional application No. 63/417,633, filed on Oct. 19, 2022.

(51) Int. Cl. A61F 2/16; A61F 9/007

(52) U.S. Cl. CPC A61F 2/167; A61F 9/00736

(57) ABSTRACT

Fluid delivery devices and methods for the treatment of ocular disorders requiring targeted and controlled administration of a fluid to an interior portion of the eye for reduction or prevention of symptoms of the disorder. The device can include a surgical instrument coupled to the distal end for an ophthalmic procedure and can have a lumen configured to convey the substance into an intraocular site of a patient. A chamber can be internal to the device or coupled to the device and configured to hold a substance. A rotary pump mechanism in the device can be configured to transfer fluid about its axis to draw the substance from the chamber and eject the substance through the lumen of the tool. A push button can be on the side of the housing and configured to engage the rotary pump to actuate a forward fluid flow towards the distal end of the device. The chamber may house continuous tubing with a rotor within the tubing creating peristaltic fluid motion, or the chamber may be sealed and fluid-filled, with a rotor acting as an impeller to move fluid to the distal end and surgical instrument.

### RELATED APPLICATIONS

The present application claims priority to prior U.S. provisional Ser. No. 63/417,633, filed Oct. 19, 2022.

### FIELD OF THE INVENTION

The present application is directed to a hand-operated (i.e., manual) intraocular fluid delivery device.

### BACKGROUND OF THE INVENTION

Minimally invasive surgical procedures involving ocular incisions and/or intraocular fluid injection can be useful for treating glaucoma and other eye conditions. For example, in a Trabeculectomy, a surgeon can use an ophthalmic blade inserted through an incision in the eye to remove a portion of the trabecular meshwork, thereby improving outflow of the aqueous humour (AH) and relieving intraocular pressure contributing to glaucoma.

During the removal of the trabecular meshwork using an ophthalmic blade, some cases have been observed in which minor bleeding occurs. When bleeding occurs during the surgery, the blood can cover up the trabecular meshwork and Schlemm's canal, creating a visual obstruction. One method of dealing with the blood reflux involves removing the ophthalmic blade from the eye and inserting a viscoelastic syringe. The viscoelastic syringe can be used to push viscoelastic into Schlemm's canal and/or into the collecting channels or move the blood away from the trabecular meshwork. Once the blood is pushed away from the trabecular meshwork, the viscoelastic syringe is removed from the eye and ophthalmic blade is re-inserted to continue the surgery.

With ophthalmic surgeries and drug deliveries in the eye, there is a need for extreme delicacy and precision by the doctor. Any advantage that a medical device can offer in terms of useability, control and precision of movements has a significant impact on surgical outcomes. Using a syringe to dispense fluid in these procedures is proving inadequate and many doctors and medical device companies have tried to find alternate means.

### SUMMARY OF THE INVENTION

This application presents fluid delivery devices and methods for the treatment of ocular disorders requiring targeted and controlled administration of a fluid into the eye for reduction or prevention of symptoms of the disorder.

A fluid injection device can include a surgical instrument coupled to the distal end for an ophthalmic procedure and can have a lumen configured to convey the substance into an intraocular site of a patient. A fluid compartment can be internal to the device or coupled to the device and configured to hold a substance.

Current fluid injection devices have drawbacks including entrapping air in the system, fluid seeping which leads to unintentional discharge, and inadequacy dealing with the high pressures often experienced. A commonly used fluid in ophthalmic surgeries, viscoelastic, is nonnewtonian and extremely viscous. The characteristics of viscoelastic when used through a reciprocating pump are the cause of poor performance in both device output, as well as the high user force required to dispense fluid. A peristaltic design pump, or externally deforming and advancing fluid through a flexible tube, is superior for dispensing these types of fluids. The fluid path can be a continuous flexible tube which eliminates air entrapment and is better for advancing even the most viscous fluids through the system.

A peristaltic pump system contained in a handheld device can provide the control and precision the doctor requires without the performance limitations of alternative pump systems.

Alternatively, a rotary pump mechanism in the device can be configured to transfer fluid about its axis to draw the substance from the fluid compartment and eject the substance through the lumen/flexible tubing of the tool. One benefit of a rotary pump, like a peristaltic pump, is that fluid can be drawn out of a reservoir. This reservoir can be an internal chamber, or an externally attached chamber, such as a syringe.

Pressing the handle of the pump causes the mechanism to rotate, causing the rollers to crush/squeeze the tubing (Durometer Shore80A or less) which moves the fluid though the tubing and into the cannula to be delivered to the patient. Upon initial use, the handle should be pressed enough times so that fluid is dispensed from the distal end prior to use on the patient.

No part of the mechanism makes contact with the fluid itself. Theoretically, this device could achieve the lowest biocompatibility risk of any ophthalmic pump since the fluid only needs to contact the lumen/flexible tubing and the cannula. However, more components may be necessary in the fluid path for the tubing-to-cannula connection and the tubing-to-fluid compartment connection.

A ratchet, clutch, and one-way bearing may be utilized to prevent the mechanism from reversing direction which reduces risk to the patient. The nature of a manually powered rotary pump also lowers the risk of delivering excess fluid to the patient because there is little pressure buildup within the fluid as compared with other standard fluid pumps.

A push button can be on the side of the housing and configured to engage the rotary pump to actuate a forward fluid flow towards the distal end of the device.

Stored Energy

With a direct drive design, as the handle is depressed the peristaltic pump, which is directly linked, moves a corresponding amount. The speed and length of handle depression directly correlates to fluid output.

With a stored energy design, there is a kinetic force driving the pump. When depressing the handle, it acts as a trigger to release a catch and expend some of the kinetic energy to independently rotate the pump.

Stored energy in the form of a spring can come from a wound clock spring supplying a rotary force to the pump. Stored energy in the form of a spring can also come from a linear spring exerting force on a rack which turns a pinion to supply rotary force to the pump. These stored energy methods can come preloaded or can be loaded by the end user. An example of this would be the user winding up the clock spring or “cocking” the linear spring.

The catch mechanism could be preset or configurable by the user. This catch mechanism would allow escapement of the rotary pump while it has a rotary force applied on it. An example of this type of escapement mechanism with a rotary force applied to it is a clock. In this pump mechanism, a gear with set tooth spacing can dictate the amount of rotation per handle depression. This would allow the user to dispense a set amount of fluid with each handle depression.

This method of stored energy to drive the pump will allow a shorter handle motion along with less force. This is critical for device stability and accuracy when dispensing fluid into the eye.

While the catch mechanism is ideal for dispensing a set amount of fluid, sometimes a continuous flow is desired. When a surgeon uses a syringe to inject fluid, they often dose according to visual feedback. For example, when filling the eye with viscoelastic, they would supply enough fluid to displace all present eye fluid, remove bubbles and hold the shape of the anterior chamber based off their visual feedback.

In place of a catch mechanism, a braking mechanism can be used to supply continuous flow. When the handle is depressed, the brake is released, allowing the pump to rotate. A braking mechanism could directly constrain the axis of rotation of the pump, or it can engage the moving rack. The fluid flow path after the pump could also be pinched or contain an open/shut valve that the handle controls.

In this design, dampening can be used to set the fluid flow velocity. Dampening will allow the pump to spin at a set speed when released, rather than spin uncontrolled until the stored energy is exhausted. The mechanical drive can be dampened directly or the fluid outflow can be constricted to limit fluid flow exiting the device. An example of controlling the fluid flow rate directly would be a valve that the user can adjust to get the desired flow rate.

CO2 Design

Another variation of the rack and pinion method to supply rotational force, would be to replace the linear spring with a piston driven by compressed gas. An example of a compressed gas would be a CO2 cartridge that is already commonly sold.

CO2 cartridges are currently used to power a number of mechanical devices. Some examples include parachute deployment devices, air powered rifles, and intraocular lens deployment devices. In these cases, the gas excerpts a force on a mechanical element. With a CO2 cartridge, the CO2 is a liquid while in its container. As the liquid CO2 enters a larger space, such as a cylinder, it turns to gas. While in the expansion phase from liquid to gas, the pressure exerted from the CO2 is very constant. This constant force can be applied to a piston, so that over the length of the stroke of the piston, the force applied to the piston is very constant. A CO2 driven piston driving a rack would have a more uniform force over the length of travel as compared to a spring driving a rack, since the spring force decreases as the spring approaches relaxed length.

Turbo Design

Another method of supplying rotary force to the peristaltic pump with CO2 is to use a turbocharger like design. In this design, there is a turbine section that is connected mechanically, such as a shaft, to the rotary pump. As the CO2 passes through the turbine or impeller section, it will cause this impeller to rotate and in turn drive the rotary pump that it is coupled to. This design can accommodate both the catch mechanism or braking mechanism to control the fluid outflow.

Activating the CO2 Cartridge

With a CO2 powered device, the user could activate or charge the device by penetrating the cartridge. As with current CO2 devices on the market, this activation could be accomplished by a twisting, pressing or lever mechanism by the user.

Electric Motor Drive

Another method of supplying rotary force to the peristaltic pump is with an electric motor. The most widely available industrial peristaltic pumps are commonly driven by an electric motor. In these commercially available designs, the operator either loads tubing into the pump body or connects tubing to inlet and outlet ports on the pump. These pumps are usually large and made to sit on a desk, or be attached to a power tool.

In this design, the peristaltic pump and driving motor are both be scaled down to fit inside of a handset for ophthalmic use. The motor could be driven by an onboard power source or coupled to external power. Internalizing these components limits the tubing running out of the device and allows for freedom of movement. It also eliminates the large pump unit that would have to sit in the operating room and possibly inside of the sterile field. By eliminating the external pump unit, the length of travel for the ophthalmic fluid is decreased and therefore reduces waste. Ophthalmic fluid, such as viscoelastic, can be very expensive and often only sold in small vials.

A motor driven pump allows the use of electrical controls which can be set by the user to dictate the fluid output. The user can select a specific dosage per handle depression or select a continuous flow with adjustable flow rate.

### BRIEF DESCRIPTION OF THE DRAWINGS

Features and advantages of the present invention will become appreciated as the same become better understood with reference to the specification, claims, and appended drawings wherein:

FIG. 1 is a side view of a device for controlled administration of a fluid to an interior portion of the eye having a handle and internal manually-powered rotating peristaltic pump that splits incoming fluid into segments and drives those segments forward to effect fluid transfer from a source of fluid through a cannula mounted at a distal end of the handle;

FIG. 2 is a cross sectional perspective view of FIG. 1 and shows the internal mechanisms and an exposed peristaltic pump assembly;

FIG. 3 is an alternative design to FIG. 2 wherein the distal cannula is a detachable unit;

FIG. 4 shows the fluid component path of FIG. 1 without an outer housing;

FIG. 5 is an exploded view of FIG. 3;

FIG. 6 is a perspective isolated view of the roller assembly;

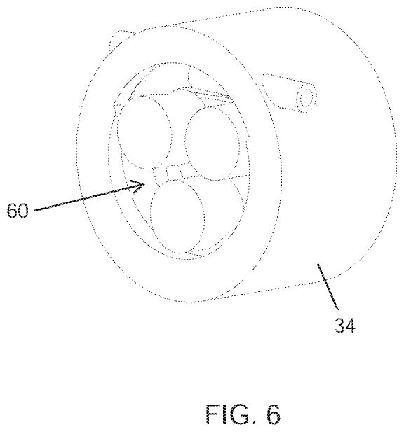

FIG. 7 is an exploded view of FIG. 6 with labeled components;

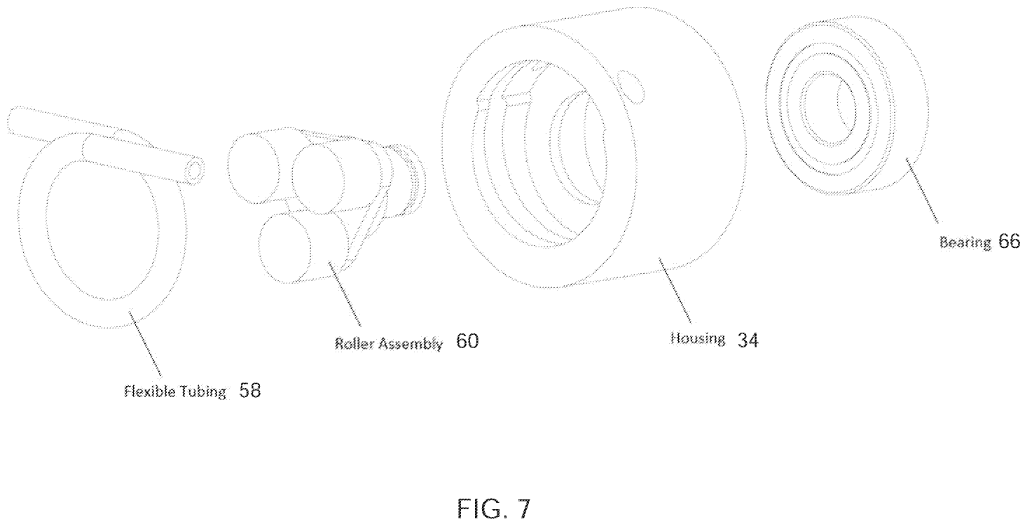

FIGS. 8A and 8B demonstrate how the mechanism of FIG. 1 rotates the roller assembly when force is applied to the end of the handle;

FIGS. 9A-9C demonstrate how the roller assembly may advance each time the handle is pressed;

FIG. 10 is an alternative design of FIG. 1 in a cross-sectional perspective view with a spring wiper assembly replacing the roller assembly;

FIGS. 11A-11C demonstrate how the wiper assembly may advance each time the handle is pressed, released, and allowed to reset;

FIG. 12 is an exploded view of the fluid path of the device in FIG. 10 with labeled components;

FIGS. 13A-13C demonstrate how the wiper assembly may advance each time the handle is pressed;

FIG. 14 is an alternative design of FIG. 1 in a cross-sectional perspective view with a star-shaped impeller with flexible vanes replacing the roller assembly;

FIG. 15 shows the rotational direction of the wiper wheel with each spoke representing a stage in rotation;

FIG. 16 is an exploded view of the fluid path of the device in FIG. 14 with labeled components;

FIG. 17 demonstrates how a syringe may be attached to the back to let fluid flow through the system; and

FIG. 18 is an alternative design of FIG. 17 where fluid is collected into an internal reservoir before dispensing.

### DETAILED DESCRIPTION OF THE PREFERRED EMBODIMENTS

The present application provides fluid delivery devices and methods for the treatment of ocular disorders requiring targeted and controlled administration of a fluid to an interior portion of the eye for reduction or prevention of symptoms of the disorder. Applications may include: injecting viscoelastic into the SC, injecting mitomycin under the conjunctiva, and medicine delivery via intravitreal injection.

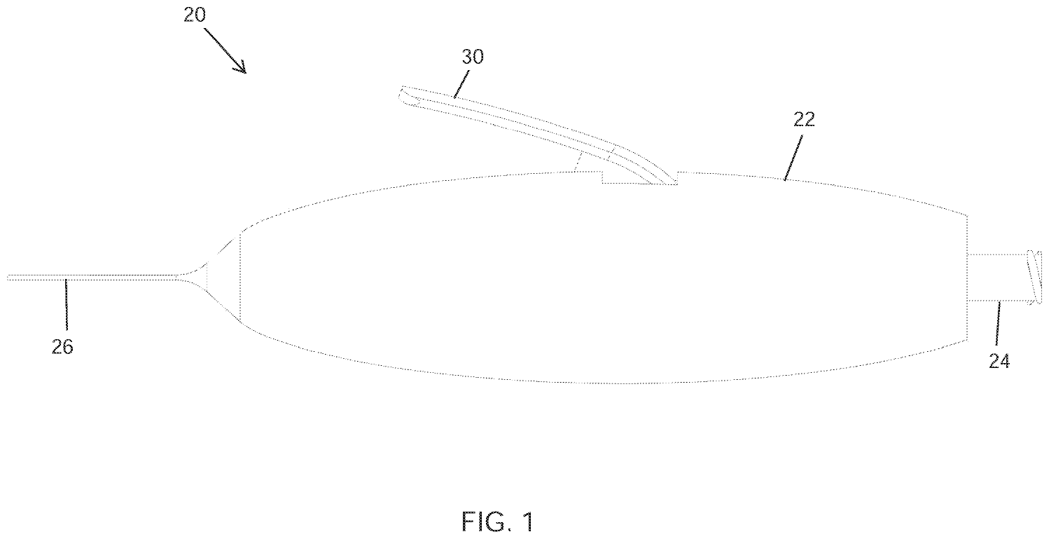

FIG. 1 is a side view of a device 20 for controlled administration of a fluid to an interior portion of the eye having a handle and internal manually-powered rotating peristaltic pump that splits incoming fluid into segments and drives those segments forward to effect fluid transfer from a source of fluid through a cannula mounted at a distal end of the handle.

The device 20 has an outer housing 22 with a proximal luer fitting 24 and a distal surgical instrument 26. The instrument 26 may be a needle, a bent or straight tube, or another specially designed part to interface with selected tissue. In the illustrated embodiment, the instrument 26 is a straight cannula having a distal tip adapted for insertion into an intraocular space in an eye. The device 20 further includes an actuator 30 for advancing fluid in a proximal-distal direction through the device.

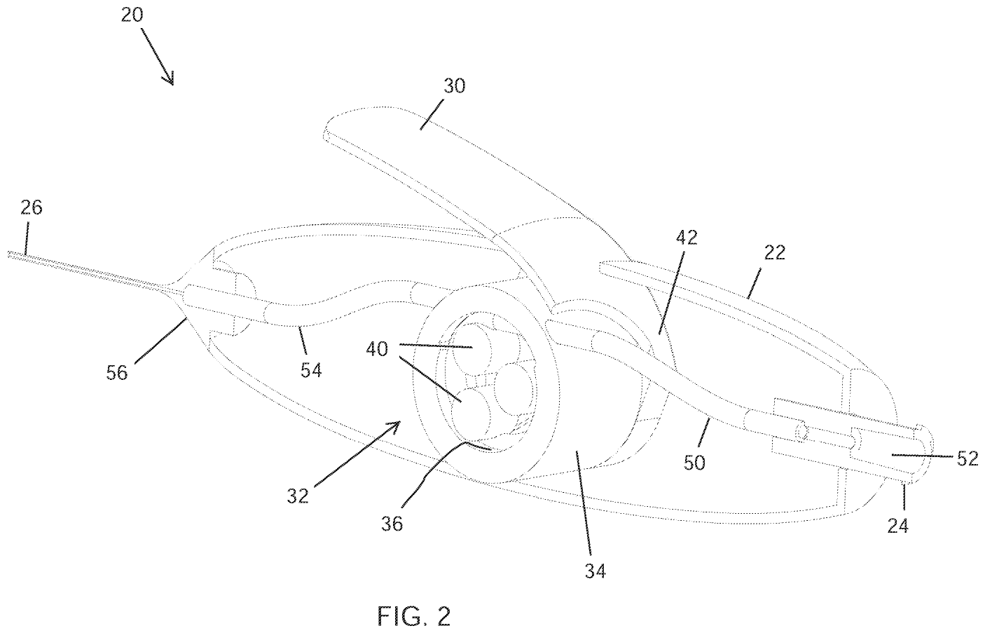

FIG. 2 is a cross sectional perspective view of FIG. 1 and shows the internal mechanisms and an exposed peristaltic pump assembly 32. The assembly 32 includes a hollow chamber 34 mounted within the outer housing 22 and defining an inner wall 36. A plurality, preferably three, of rollers 40 are mounted for rotation within the chamber 34 and form a rotor. The actuator 30 is connected to a lever 42 or other such device which causes the three rollers 40 to rotate.

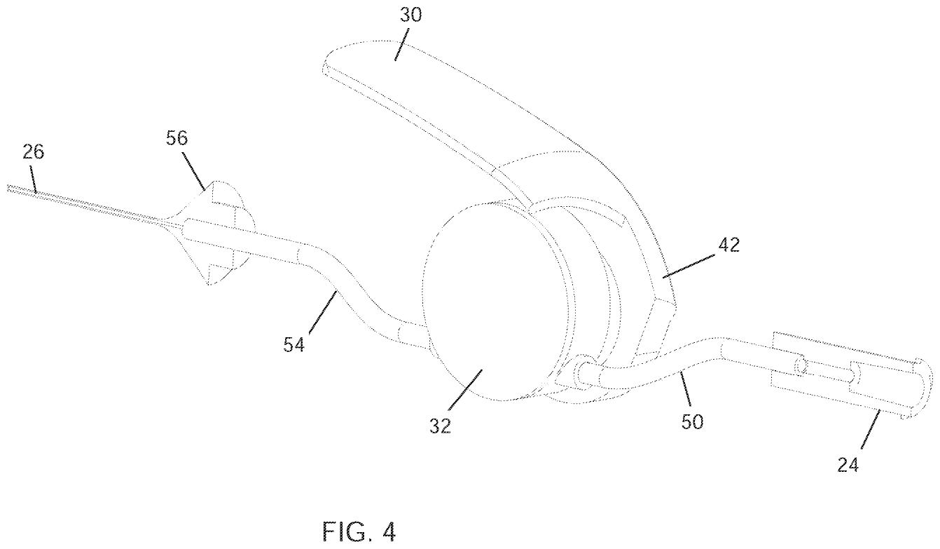

Flexible inlet tubing 50 connects to a fluid inlet 52 defined by the proximal luer fitting 24 and extends to the chamber 34. Flexible outlet tubing 54 connects to a fluid outlet 56 defined by a proximal end of cannula 26 and extends to the chamber 34. The inlet tubing 50 and outlet tubing 54 in the illustrated embodiment are contiguous and define a helical loop 58 around the inner wall 36 of the chamber 34. FIG. 4 shows the fluid component path without the outer housing 22. Rotation of the rollers 40 creates peristaltic movement of fluid within the tubing that extends through the device 20, as will be seen below.

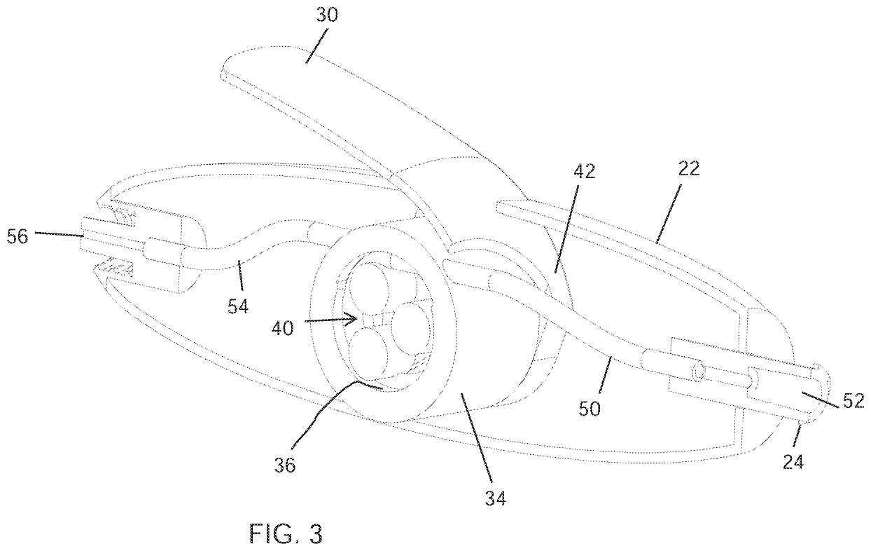

FIG. 3 is an alternative design of the device 20 of FIG. 2 wherein the distal instrument (not shown) is provided as a detachable unit that connects to the fluid outlet 56 by, for example, mating threading.

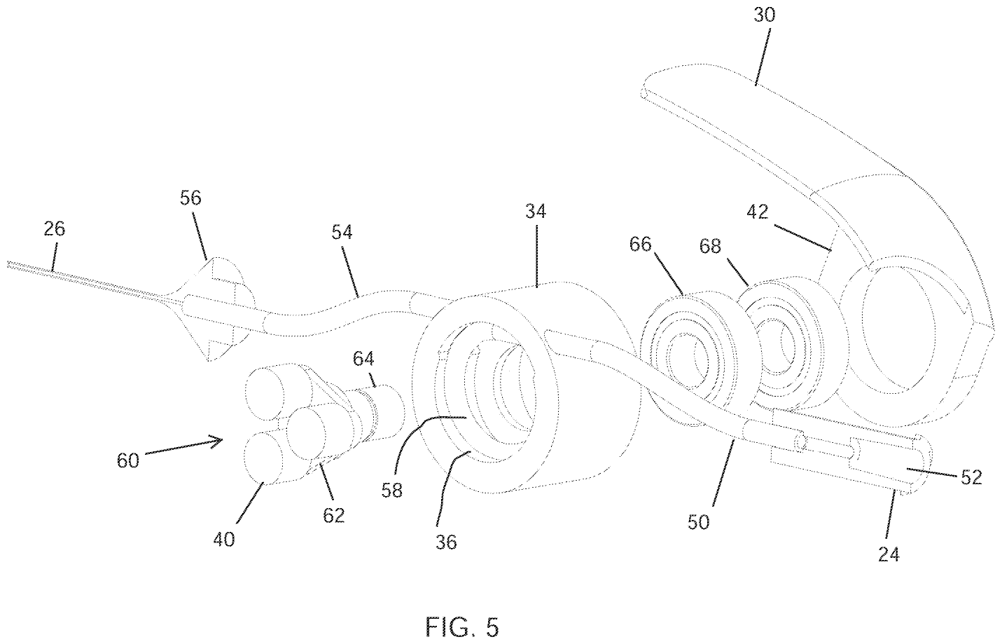

FIG. 5 is an exploded view of FIG. 3 and shows the rotor 60 comprising the three rollers 40 mounted on a common plate 62 and having a shaft stub 64 extending therefrom. The helical loop 58 of tubing is also shown in better detail. When assembled, the rollers 40 are aligned with the helical loop 58 of tubing and the shaft stub 64 extends into a through bore of at least a first bearing 66. The lever 42 of the actuator 30 may be formed in a ring shape that mounts to a second bearing 68. Although not shown, a ratcheting mechanism is provided such that the pressing the actuator 30 rotates the rotor 60 in only one direction. That is, mating teeth or a pawl and tooth assembly is provided between the lever 42 and the rotor 60 such that the pressing the actuator 30 rotates the rotor, while releasing the actuator allows the mechanism to return to its original position without reversing the rotation of the rotor.

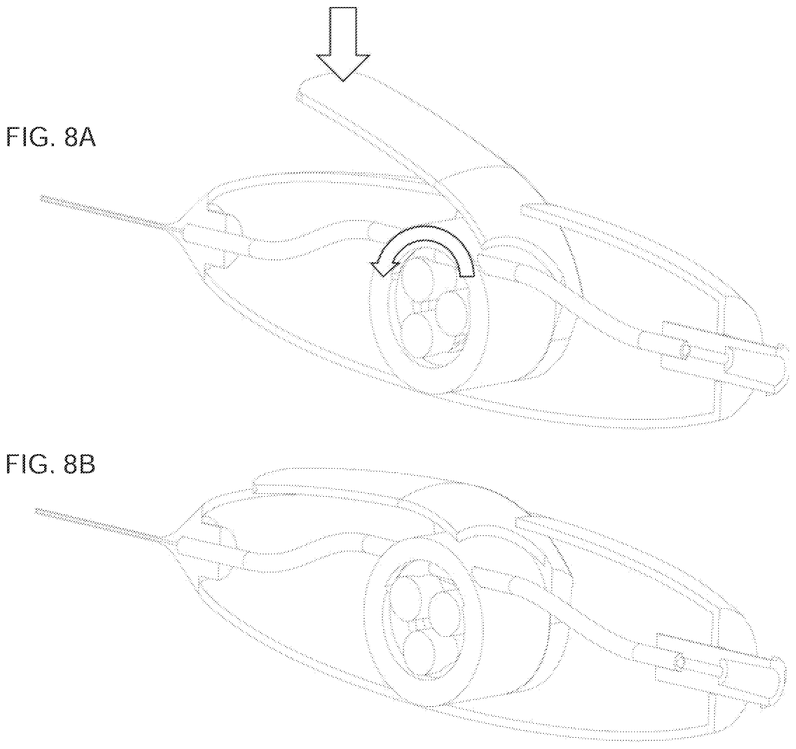

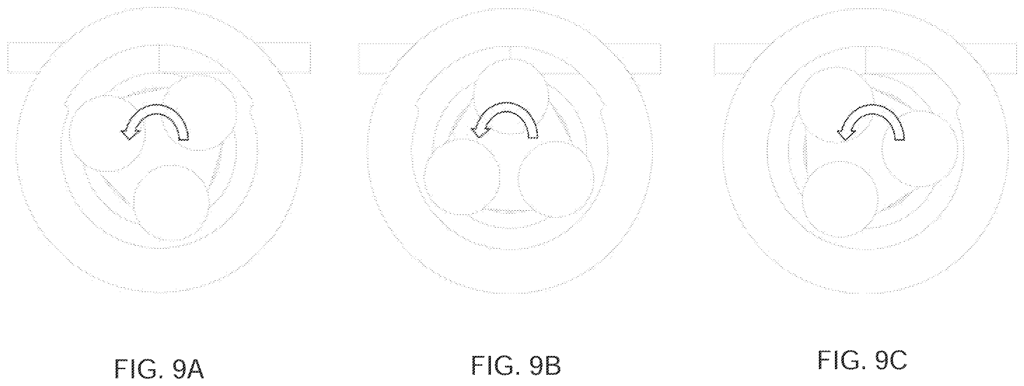

FIGS. 8A and 8B demonstrate how the mechanism of FIG. 1 rotates the roller assembly when force is applied to the end of the handle, and FIGS. 9A-9C demonstrate how the roller assembly may advance each time the handle is pressed.

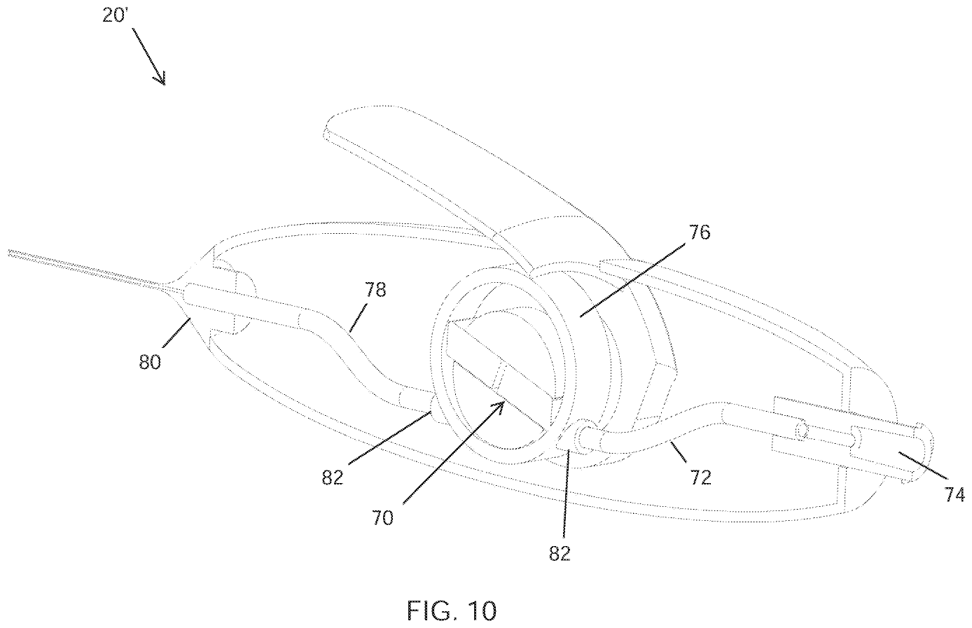

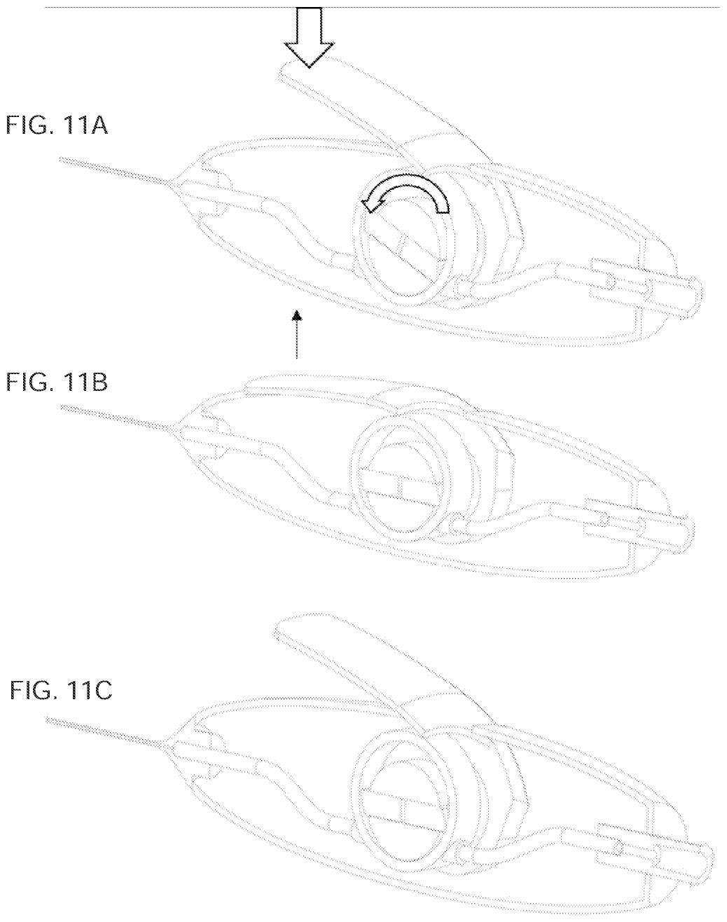

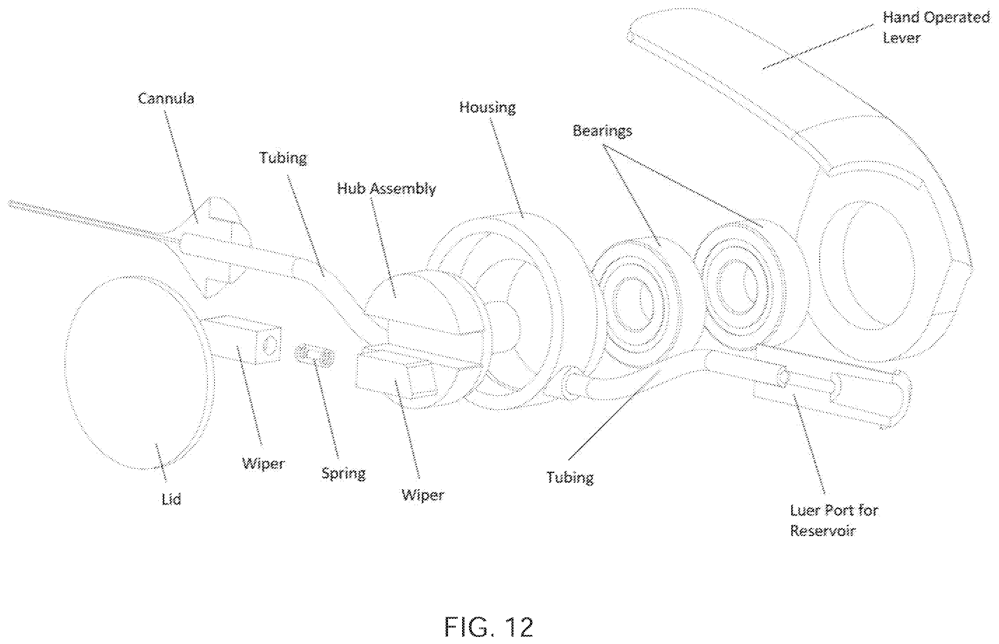

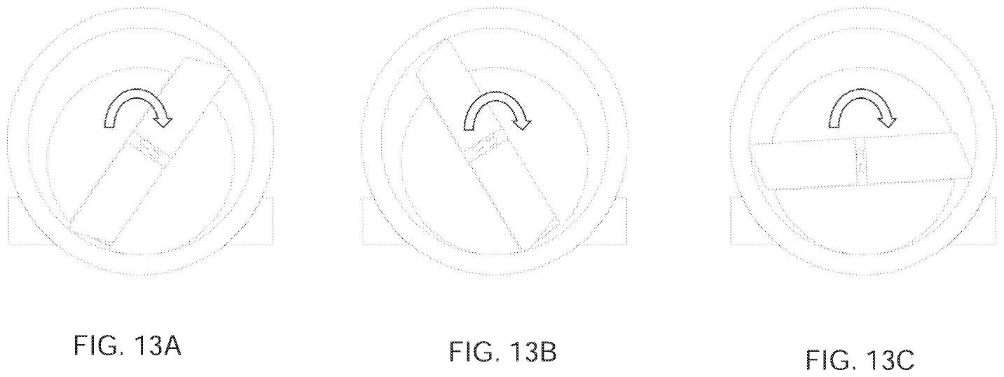

FIG. 10 is an alternative design of a device 20′ such as in FIG. 1 in a cross-sectional perspective view with a spring wiper assembly 70 replacing the rotor assembly 60. FIGS. 11A-11C demonstrate how the wiper assembly 70 may advance each time the handle is pressed, released, and allowed to reset. FIG. 12 is an exploded view of the fluid path of the device in FIG. 10 with labeled components. FIGS. 13A-13C demonstrate how the wiper assembly 70 may advance each time the handle is pressed.

In the embodiment of FIGS. 10-13, flexible inlet tubing 72 connects to the fluid inlet 74 and extends to the chamber 76, while flexible outlet tubing 78 connects to the fluid outlet 80 and extends to the chamber. Both inlet and outlet tubing 72, 78 terminate at fittings 82 extending through the wall of the chamber 76. The interior of the chamber 76 is sealed such that fluid may be provided through the inlet tubing 72 to fill the chamber. As the wiper assembly 70 rotates in one direction, the wipers therein propel fluid in a proximal-distal direction through the device 20′. All other aspects and options are similar to those described above with respect to the first embodiment.

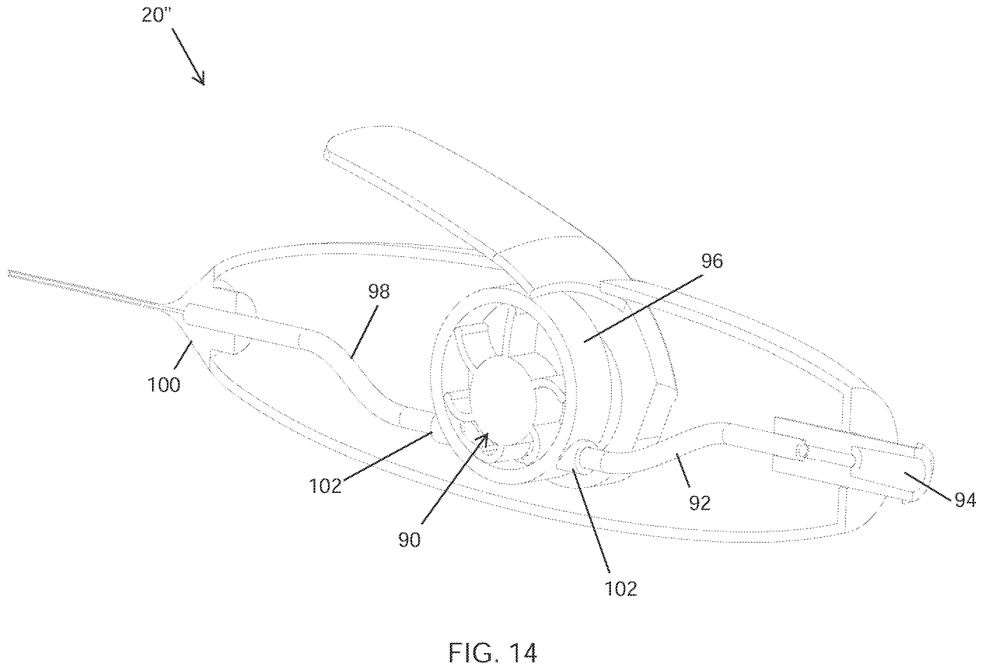

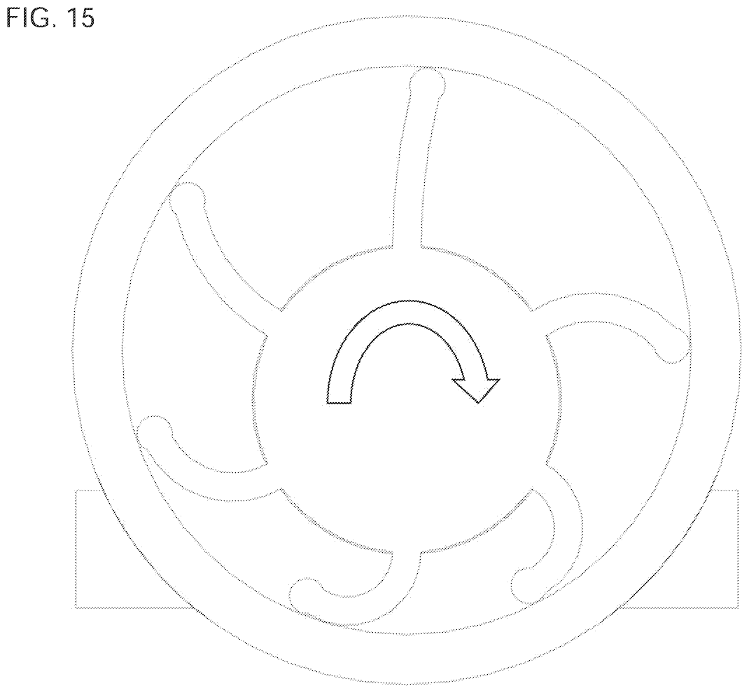

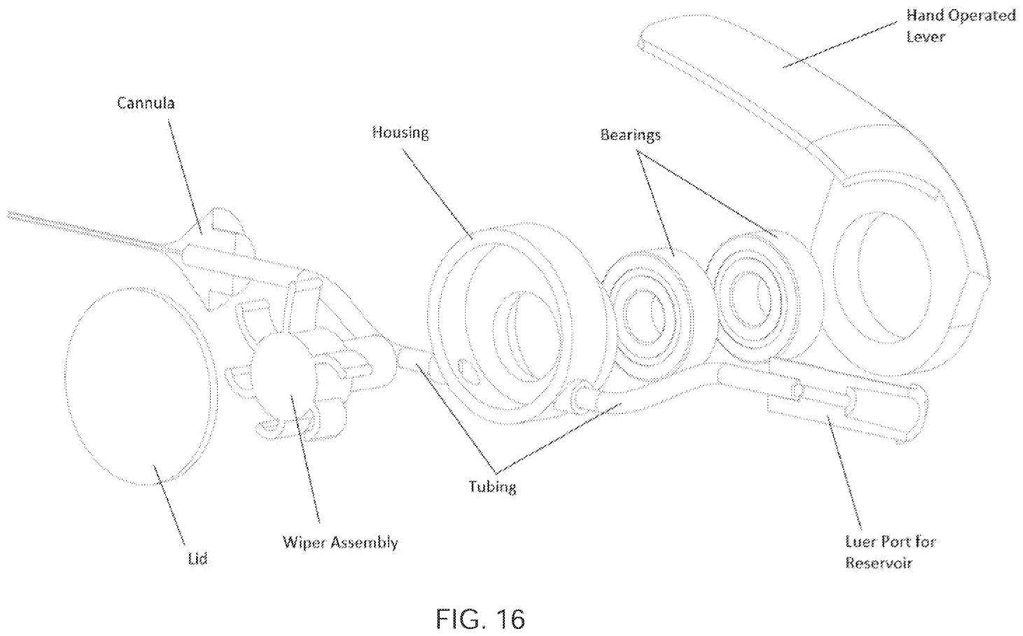

FIG. 14 is a second alternative of a device 20″ such as in FIG. 1 in a cross-sectional perspective view with a star-shaped impeller 90 with flexible spokes or vanes replacing the roller assembly. FIG. 15 shows the rotational direction of the wiper wheel 90 with each spoke representing a stage in rotation. FIG. 16 is an exploded view of the fluid path of the device in FIG. 14 with labeled components.

In the embodiment of FIGS. 14-16, flexible inlet tubing 92 connects to the fluid inlet 94 and extends to the chamber 96, while flexible outlet tubing 98 connects to the fluid outlet 100 and extends to the chamber. Both inlet and outlet tubing 92, 98 terminate at fittings 102 extending through the wall of the chamber 96. The interior of the chamber 96 is sealed such that fluid may be provided through the inlet tubing 92 to fill the chamber. As the wiper assembly 90 rotates in one direction, the wipers therein propel fluid in a proximal-distal direction through the device 20″. All other aspects and options are similar to those described above with respect to the first embodiment.

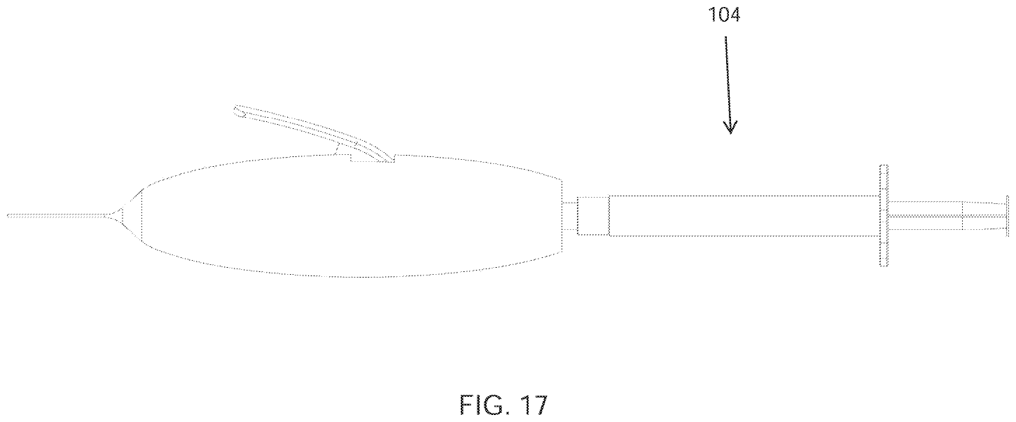

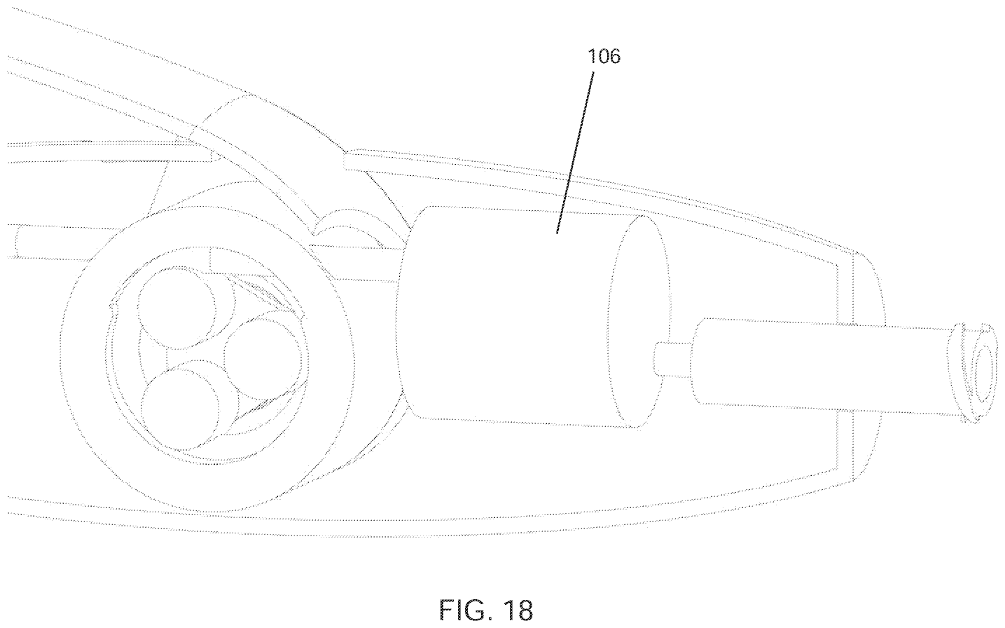

FIG. 17 demonstrates how a syringe 104 may be attached to the back to let fluid flow through the system. FIG. 18 is an alternative design of FIG. 17 where fluid is collected into an internal reservoir 106 before dispensing.

Aspects of the present application may include:

Fluid drawn from internal or external reservoir.

Fluid enters eye through a body that penetrates the cornea/sclera and delivers fluid:
a. From the distal end of body
b. From side holes of body
c. From a body that is curved to match tissue curvature
d. From a secondary fluid delivery body that is deployed from the main body (e.g. a catheter or actuated tip)
e. An application of the design would be to use the device for a canaloplasty via viscoelastic fluid delivery. A primary or secondary fluid delivery body may sit against, partially inside, or fully inside the TM/SC to deliver viscoelastic through the SC.
f. An alternate design is to have a detachable connection port at the end of the pump (such as a luer connection) to allow the practitioner to select an appropriate apparatus to deliver fluid. These apparatuses may include a needle, a bent or straight tube, or a specially designed part to interface with selected tissue.

Fluid may be transferred by a pump that works by splitting the fluid into segments and then driving those segments forward in a rotary fashion.
a. Fluid can be transferred in a flexible tube where an external roller/wiper presses on the tube to constrict it, while advancing along the tube to move the fluid, in a peristaltic manner, or
b. Fluid can be split into chambers, where a wiper advances the fluid and a changing chamber size forces the liquid to advance out of the pump. The wiper may be a star-shaped impeller having flexible vanes, and
c. Alternatively, where the fluid path (e.g. flexible tubing or wiper assembly) can be replaced and disposed of, while other non-fluid path components are reusable.

The application also discloses a rotary pump mechanism configured to drive fluid in one direction with the aid of a ratchet, clutch, and one-way bearing.
a. An alternate design is to have a switch to allow the user to specify which direction the rotation/pumping is occurring.
   i. This is similar to the switch on a ratchet wrench to change direction.
   ii. In this case, the pumping rotation can turn into a vacuum to draw fluid/blood out of a specific area.
b. The design may include one or more ratchet mechanisms.
   i. Similar to tightening a bolt with a ratchet wrench, a certain amount of friction is needed to allow the ratcheting to advance. In a low friction design, a second ratcheting mechanism can be inserted to allow both ratches to alternately lock onto a shaft and spin the shaft in one direction only.

The rotation of the pump will be controlled and mechanically driven via a manually operated arm/lever.
a. An alternate design is to have a mechanism (such as gears) to increase or decrease the rotation amount of torque applied versus a straight arm/lever.

The practitioner will interface with this arm directly to drive the pump. The actuation of this arm is to be of a size, force and movement such that it may be operable with a finger while holding the device.

While the invention has been described in its preferred embodiments, it is to be understood that the words which have been used are words of description and not of limitation. Therefore, changes may be made within the appended claims without departing from the true scope of the invention.

What is claimed is:

1. A hand-operated manual intraocular fluid delivery device, comprising:
a handle having an outer housing and a chamber located within the housing, the housing having a distal end with a fluid outlet and a fluid inlet;
flexible inlet tubing connected to the fluid inlet and extending to the chamber and flexible outlet tubing connected to the fluid outlet and extending to the chamber;
a cannula having a distal tip adapted for insertion into an intraocular space in an eye and a proximal end in fluid communication with the fluid outlet;
a lever mounted in the housing for reciprocating motion and having an outer manually-operated actuator; and
a rotor arranged to rotate within the chamber and configured to be rotated by the lever, wherein
manually operating the actuator causes the rotor to advance a measure of fluid from the inlet tubing through the chamber to the outlet tubing, and from there through the fluid outlet and through the cannula distal tip into the intraocular space.

2. The device of claim 1, wherein the chamber houses continuous tubing and the rotor compresses the tubing to create peristaltic fluid motion, or the chamber may be sealed and fluid-filled, with a rotor acting as an impeller to move fluid to the distal end and surgical instrument.

3. The device of claim 1, wherein the chamber is sealed and fluid-filled, and the rotor has a wiper that acts as an impeller to move fluid to the fluid outlet.
